(19) United States
(12) Patent Application Publication
(10) Pub. No.: US 2006/0199958 A1
(43) Pub. Date: Sep. 7, 2006

(54) Process and intermediates for the preparation of pyrrolidine carboxylic acids

(76) Inventors: Raymond Cvetovich, Scotch Plains, NJ (US); John Y. Chung, Edison, NJ (US); Joseph S. Amato, Millerton, NY (US); Lisa Dimichele, Berkeley Heights, NJ (US)

Correspondence Address: MERCK AND CO., INC, P O BOX 2000, RAHWAY, NJ, 07065-0907, US

(21) Appl. No.: 10/550,640

(22) PCT Filed: Apr. 9, 2004
(86) PCT No.: PCT/US04/11253

§ 371 Date: Sep. 23, 2005

Related U.S. Application Data
(60) Provisional application No. 60/462,795, filed on Apr. 14, 2003.

(51) Int. Cl. C07D 417/02; C07D 413/02; C07D 403/02

(52) U.S. Cl. 544/238; 544/333; 544/405; 546/279.1; 548/200; 548/215; 548/311.1; 546/148; 546/176; 548/517; 548/518; 548/465; 548/530; 548/305.4

(57) ABSTRACT

A novel process is provided for the preparation of pyrrolidine carboxylic acids, and the useful intermediates obtained therein. These compounds are intermediates for the synthesis of melanocortin-4 receptor (MC-4R), which are useful for the treatment of disorders such as obesity, diabetes, sexual dysfunction, male sexual dysfunction, and female sexual dysfunction.

### BACKGROUND OF THE INVENTION

The present invention provides a process for the preparation of pyrrolidine carboxylic acids of general formula (I).

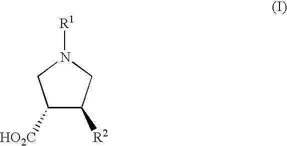

The present invention also provides intermediates useful in the disclosed process.

The compounds of formula (I) are intermediates useful for the preparation of the pyrrolidine compounds of the general formula (II), wherein R₂ is phenyl, unsubstituted or substituted with one to three R₃ groups, r is 1 and s is 1.

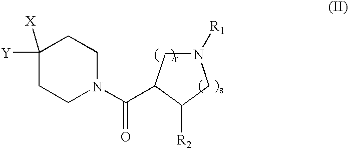

The compounds of formula (II), along with their use as melanocortin receptor agonists were disclosed in WO 02/068387 (published on Sep. 6, 2002), and WO 02/068388 (published on Sep. 6, 2002). The compounds of formula (II) are also useful as agents for the treatment, control or prevention of diseases, disorders or conditions responsive to the activation of one or more of the melanocortin receptors including, but are not limited to, MC-1, MC-2, MC-3, MC-4, or MC-5. Such diseases, disorders or conditions include, but are not limited to, obesity, diabetes mellitus, hypertension, hyperlipidemia, osteoarthritis, cancer, gall bladder disease, sleep apnea, depression, anxiety, compulsion, neuroses, insomnia/sleep disorder, substance abuse, pain, male and female sexual dysfunction, fever, inflammation, immune modulation, rheumatoid arthritis, skin tanning, acne and other skin disorders, neuroprotective and cognitive and memory enhancement including the treatment of Alzheimer's disease. Some compounds encompassed by formula (II) show highly selective affinity for the melanocortin-4 receptor (MC-4R) relative to MC-1R, MC-2R, MC-3R, and MC-5R, which makes them especially useful in the prevention and treatment of obesity, as well as male and/or female sexual dysfunction, including erectile dysfunction.

WO 02/068387 and WO 02/068388 describe processes for preparing the compounds of formula (II). However, the pyrrolidine acid was prepared in racemic forms and required a chiral HPLC chromatography. This resulted in the loss of all of the material prepared as the wrong enantiomer.

The present invention is directed to an efficient chiral synthesis that produces a pyrrolidine acid of structural formula (I) in a higher yield and utilizes less expensive chemical reagents. The synthetic sequence comprises 5 steps with an overall yield of about 71% and a chiral purity of >99.9% ee of the pyrrolidine acid without the use of chromatography.

The synthesis of phenyl- and benzyl-substituted racemic pyrrolidines by intramolecular C-alkylation is described in Achini, R., Helvetica Chimica Acta, 64, 2203-2218 (1981). The asymmetric reduction of aryl chloromethyl-ketones is described in using (S)-MeCBS is described in Burkhardt, E. R. Tetr. Lett. 38, 1523-1526 (1997). The asymmetric transfer hydrogenation of ring-substituted 2-chloroacetophenone is reported by Noyori, et al., Org. Lett, 4, 4373 (2002). The reduction of 2-chloro-2′,4′-difluoroacetophenone with NaBH₄/Me₃SiCl catalyzed by (S)-α,α-diphenylpyrrolidinemethanol to give chlorohydrins is described in Jiang et al., Tetr. Lett., 41, 10281-10283 (2000). The rate acceleration of the Michael addition of tertiary amines to acrylonitrile using a polar solvent is disclosed in Aggarwal, V. et al., J. Org. Chem. 67, 510-514 (2002).

### SUMMARY OF THE INVENTION

This invention is concerned with a process for preparing compounds of structural formula (I) and certain useful intermediates obtained during that process.

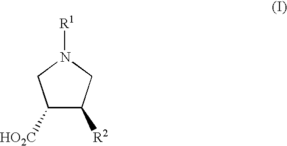

The novel process and novel intermediates can be exemplified in Scheme A, which shows the preparation of pyrrolidine acid (I).

The process involves the chiral reduction of the halogenated ketone (IV) to form a halogenated alcohol (V). The halogenated alcohol (V) is then converted to the amino alcohol (VII), via the epoxide intermediate (VI), by treatment with a base and subsequent treatment with a primary amine. The conjugate addition of the resulting amino alcohol (VII) to an α,β unsaturated nitrile or ester (Y═—CN or —CO₂R⁵, and R⁵ is C₁₋₄ alkyl) affords the tertiary amine (VII). The alcohol of compound (VIII) is then converted to a leaving group (shown as —OZ in intermediate IX) by treatment with an alcohol activating reagent, such as ClPO(OR⁶)₂, ClPO(N(R⁶)₂)₂, MsCl, Ms₂O, TsCl or Ts₂O. The resulting intermediate (IX) is then treated with a base to facilitate the intramolecular cyclization to give a cis/trans mixture of pyrrolidine (X). The Y group of pyrrolidine (X) is then hydrolyzed/epimerized give the trans pyrrolidine acid (I).

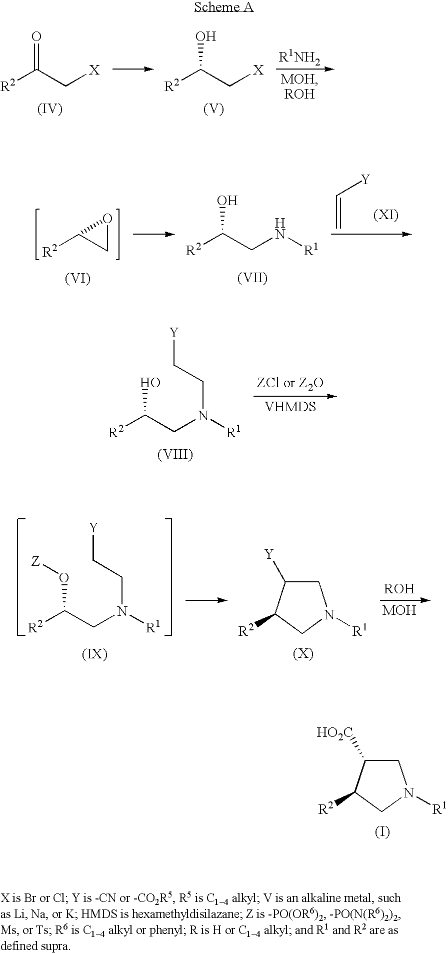

Also provided are intermediate compounds which are useful for the preparation of compounds of structural formula (I).

### DETAILED DESCRIPTION OF THE INVENTION

The present invention provides a process for the preparation of compounds of structural formula (I): 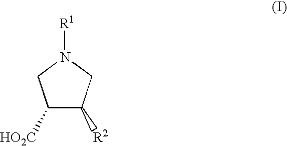
wherein
R¹ is selected from the group consisting of
(1) hydrogen,
(2) amidino,
(3) C₁₋₄ alkyliminoyl,
(4) C₁₋₁₀ alkyl,
(5) —(CH₂)ₙ—C₃₋₇ cycloalkyl,
(6) —(CH₂)ₙ-phenyl,
(7) —(CH₂)ₙ-naphthyl, and
(8) —(CH₂)ₙ-heteroaryl,
   in which phenyl, naphthyl, and heteroaryl are unsubstituted or substituted with one to three groups independently selected from R³; and alkyl, cycloalkyl, and (CH₂)ₙ are unsubstituted or substituted with one to three groups independently selected from R³ and oxo;
   R² is selected from the group consisting of
(1) C₁₋₄ alkyl,
(2) —(CH₂)ₙ-cycloalkyl,
(3) —(CH₂)ₙ-heterocycloalkyl,
(4) —(CH₂)ₙ-phenyl,
(5) —(CH₂)ₙ-naphthyl, and
(6) —(CH₂)ₙ-heteroaryl wherein heteroaryl is selected from the group consisting of
   (1) pyridinyl,
   (2) furyl,
   (3) thienyl,
   (4) pyrrolyl,
   (5) oxazolyl,
   (6) thiazolyl,
   (7) imidazolyl,
   (8) pyrazolyl,
   (9) isoxazolyl,
   (10) isothiazolyl,
   (11) pyrimidinyl,
   (12) pyrazinyl,
   (13) pyridazinyl,
   (14) quinolyl,
   (15) isoquinolyl,
   (16) benzimidazolyl,
   (17) benzofuryl,
   (18) benzothienyl,
   (19) indolyl,
   (20) benzthiazolyl, and
   (21) benzoxazolyl;
      in which alkyl, phenyl, naphthyl, heteroaryl, and (CH₂)ₙ are unsubstituted or substituted with one to three groups independently selected from R³;
      each R³ is independently selected from the group consisting of
(1) C₁₋₆ alkyl,
(2) —(CH₂)ₙ-phenyl,
(3) —(CH₂)ₙ-naphthyl,
(4) —(CH₂)ₙ-heteroaryl,
(5) —(CH₂)ₙ-heterocycloalkyl,
(6) —(CH₂)ₙC₃₋₇ cycloalkyl,
(7) halogen,
(8) OR⁴,
(9) —(CH₂)ₙN(R⁴)₂,
(10) NO₂,
(11) —(CH₂)ₙNR⁴SO₂R⁴,
(12) —(CH₂)ₙSO₂N(R⁴)₂,
(13) —(CH₂)ₙS(O)ₚR⁴,
(14) CF₃,
(15) CR₂CF₃,
(16) OCF₃, and
(17) OCH₂CF₃;
   in which heteroaryl is as defined above; alkyl, phenyl, naphthyl, heteroaryl, cycloalkyl, and heterocycloalkyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group; each R⁴ is independently selected from the group consisting of
(1) hydrogen,
(2) C₁₋₆ alkyl,
(3) —(CH₂)ₙ-phenyl,
(4) —(CH₂)ₙ-heteroaryl,
(5) —(CH₂)ₙ-naphthyl,
(6) —(CH₂)ₙ-heterocycloalkyl,
(7) —(CH₂)ₙC₃₋₇ cycloalkyl, and
(8) —(CH₂)ₙC₃₋₇ bicycloalkyl;
   wherein alkyl, phenyl, heteroaryl, heterocycloalkyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl; and
   n is 0, 1, 2, 3 or 4;
   comprising the steps of:
   (a) preparing an alcohol of structural formula (V) 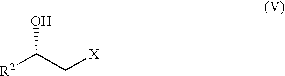
   wherein
   X is bromide or chloride, and R² is as defined above,
   by treating a ketone of structural formula (IV), 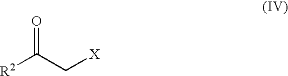
   wherein X is bromide or chloride, and R² is as defined above, with a reducing agent, and isolating the resulting product;
   (b) forming an amino alcohol of structural formula (VII) 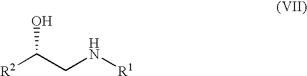
   wherein R¹ and R² are as defined above,
   by treating an alcohol of structural formula (V), wherein X is chloride or bromide and R² is as defined above, 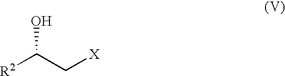
   with an amine of general formula R¹NH₂, wherein R¹ is as defined above, and a base in a solvent, and isolating the resulting product;
   (c) forming a compound of structural formula (VII) 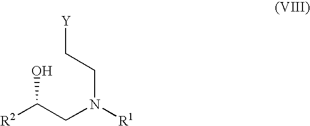
   wherein Y is —CN or —CO₂R⁵ and R⁵ is C₁₋₄ alkyl, and wherein R¹ and R² are as defined above,
   by treating the amino alcohol of structural formula (VII) 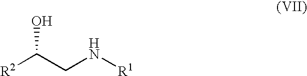
   with a compound of general formula (XI) 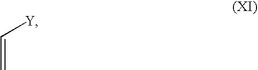
   wherein Y is —CN or —CO₂R⁵, and R⁵ is C₁₋₄ alkyl, and isolating the resulting product;
   (d) forming a pyrrolidine compound of structural formula (X) 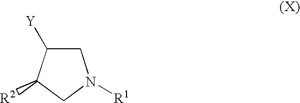
   wherein R¹ and R² are as defined above, by treating the compound of structural formula (VII), wherein Y, R¹ and R² are as defined above, 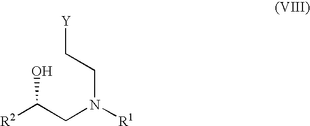
   with an alcohol activating reagent, followed by a base;
   (e) forming a trans-pyrrolidine acid of structural formula (I) 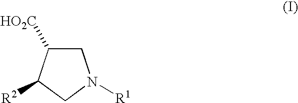
   wherein R¹ and R² are as defined above,
   by hydrolyzing the pyrrolidine compound of structural formula (X), wherein Y, R¹ and R² are as defined above, 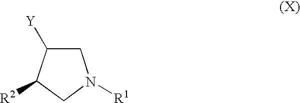
   with an aqueous base in a solvent; and
   (f) isolating the resulting product.

In one embodiment of the present invention, R² is phenyl or thienyl optionally substituted with one to three groups independently selected from R³. In a class of this embodiment, R² is phenyl optionally substituted with one to three groups independently selected from R³. In a subclass of this class, R² is selected from the group of phenyl; ortho, para-difluorophenyl; and para-methoxyphenyl. In a subclass of this subclass, R² is ortho, para-difluorophenyl.

In another embodiment, R³ is selected from the group consisting of halogen, —CF₃, and OR⁴. In a class of this embodiment of the present invention, R³ is selected from the group consisting of fluoride, bromide, chloride, —CF₃, and —OC₁₋₆ alkyl. In a subclass of this class, R³ is selected from fluoride, bromide, —CF₃, and —OCH₃.

In another embodiment, n is 0, 1 or 2. In a class of this embodiment n is 0 or 1. In a subclass of this embodiment, n is 0.

In another embodiment of the present invention, the reducing agent used to treat the compound of formula (IV) of step (a) is (+)-DIP chloride.

In another embodiment of the present invention, the compound of formula (IV) of step (a) is treated with a reducing agent in the presence of a catalyst. In a class of this embodiment the reducing agent is selected from the group consisting of borane-N,N-diethyl aniline, borane-THF, and borane-dimethylsulfide. In a subclass of this class, the reducing agent is borane-N,N-diethyl aniline. In another class of this embodiment, the catalyst is selected from the group consisting of (S)-CBS and (S)-2-methyl CBS oxazaborolidine. In a subclass of this class, the catalyst is (S)-2-methyl CBS oxazaborolidine.

In another embodiment of the present invention, alcohol of formula (V) is treated with an amine of general formula R¹NH₂, wherein R¹ is selected from the group consisting of hydrogen, —(CH₂)ₙphenyl, or C₁₋₆alkyl. In a class of this embodiment, R¹ is tert-butyl or —CH₂-phenyl. In a subclass of this class, R¹ is tert-butyl.

In another embodiment of the present invention, the alcohol of formula (V) is treated with a base selected from the group consisting of NaOH, LiOH, KOH. In a class of this embodiment, the base is NaOH.

In another embodiment of the present invention, the alcohol of formula (V) is treated in a solvent selected from methanol or ethanol. In a class of this embodiment, the solvent is methanol. In a subclass of this class, the solvent is refluxing methanol.

In another embodiment of the present invention, the amino alcohol of structural formula (VD) is isolated by recrystallization from heptane or hexane. In a class of this embodiment, the solvent is heptane.

In another embodiment of the present invention, the compound of formula (XI) is the compound wherein Y is CN.

In another embodiment of the present invention, the compound of formula (XI) is the compound wherein Y is —CO₂R⁵, wherein R⁵ is C₁₋₄ alkyl. In a class of this embodiment Y is —CO₂CH₃, —CO₂CH₂CH₃, or —CO₂CH₂CH₂CH₂CH₃. In a subclass of this class, Y is —CO₂CH₂CH₃, or —CO₂CH₂CH₂CH₂CH₃.

In another embodiment of the present invention, the compound of formula (VIII) is formed by heating the mixture to reflux.

In another embodiment of the present invention, the compound of formula (VIII) is formed by adding ethanol, formamide or a mixture thereof. In a class of this embodiment, the compound of formula (VIII) is formed by adding a 1:1 mixture of ethanol:formamide.

In another embodiment of the present invention, the compound of formula (VIII) is isolated by recrystallizing from heptane or hexane.

In another embodiment of the present invention, the compound of formula (VIII) is treated with an alcohol activating reagent selected from the group consisting of ClPO(OR⁶)₂, ClPO(N(R⁶)₂)₂, MsCl, Ms₂O, TsCl, and Ts₂O, wherein R⁶ is C₁₋₄ alkyl or phenyl. In a class of this embodiment, the alcohol activating reagent is chlorodiethyl phosphate.

In another embodiment of the present invention, the compound of formula (VIII) is treated with a base selected from the group consisting of lithium hexamethyldisilazide, sodium hexamethyl disilazide, and potassium hexamethyldisilazide. In a class of this embodiment, the base is lithium hexamethyl disilazide.

In another embodiment of the present invention, the compound of formula (VI) is treated at a temperature of about −30 to about +10 C. In a class of this embodiment, the temperature is about −15 C.

In another embodiment of the present invention, the pyrrolidine compound of formula (X) is hydrolyzed with a base selected from the group consisting of NaOH, LiOH and KOH. In one class of this embodiment, the base is NaOH. In a subclass of this class, the base is aqueous NaOH.

In another embodiment of the present invention, the pyrrolidine compound of formula (X) is hydrolyzed in a solvent selected from the group consisting of methanol, ethanol, and isopropanol. In a class of this embodiment, the solvent is ethanol.

In another embodiment, the product of step (f) is isolated by forming a zwitterion of the trans pyrrolidine acid of structural formula (I) 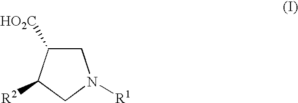
wherein R¹ and R² are as defined above, recrystallizing the zwitterion from a solvent; and isolating the resulting product.

In a class of this embodiment the zwitterion of the pyrrolidine acid of formula (I) is formed at the isoelectric pH using an acid or a base. In one subclass of this class, the acid is selected from sulfuric acid or hydrochloric acid. In a subclass of this subclass, the acid is sulfuric acid. In another subclass of this class, the isoelectric pH is about 6 and a stoichiometric amount of acid is added.

In another class of this embodiment, the zwitterion of the pyrrolidine acid of formula (I) is recrystallized from a solvent selected from the group consisting of ethanol, isopropyl alcohol, methyl tert-butyl ether or a mixture thereof. In a subclass of this class, the solvent is a mixture of isopropyl alcohol and methyl tert-butyl ether. In a subclass of this subclass, the solvent is 1:3 isopropyl alcohol:methyl tert-butyl ether.

The present invention also provides a process for the preparation of compounds of structural formula (I): 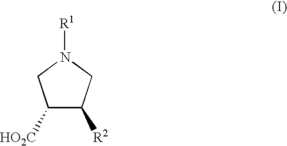
wherein
R¹ is selected from the group consisting of
(1) hydrogen,
(2) amidino,
(3) C₁₋₄ alkyliminoyl,
(4) C₁₋₁₀ alkyl,
(5) —(CH₂)ₙ—C₃₋₇ cycloalkyl,
(6) —(CH₂)ₙ-phenyl,
(7) —(CH₂)ₙ-naphthyl, and
(8) —(CH₂)ₙ-heteroaryl,
   in which phenyl, naphthyl, and heteroaryl are unsubstituted or substituted with one to three groups independently selected from R³; and alkyl, cycloalkyl, and (CH₂)ₙ are unsubstituted or substituted with one to three groups independently selected from R³ and oxo;
   R² is selected from the group consisting of
(1) C₁₋₄ alkyl,
(2) —(CH₂)ₙ-Cycloalkyl,
(3) —(CH₂)ₙ-heterocycloalkyl,
(4) —(CH₂)ₙ-phenyl,
(5) —(CH₂)ₙ-naphthyl, and
(6) —(CH₂)ₙ-heteroaryl wherein heteroaryl is selected from the group consisting of
   (1) pyridinyl,
   (2) furyl,
   (3) thienyl,
   (4) pyrrolyl,
   (5) oxazolyl,
   (6) thiazolyl,
   (7) imidazolyl,
   (8) pyrazolyl,
   (9) isoxazolyl,
   (10) isothiazolyl,
   (11) pyrimidinyl,
   (12) pyrazinyl,
   (13) pyridazinyl,
   (14) quinolyl,
   (15) isoquinolyl,
   (16) benzimidazolyl,
   (17) benzofuryl,
   (18) benzothienyl,
   (19) indolyl,
   (20) benzthiazolyl, and
   (21) benzoxazolyl;
      in which alkyl, phenyl, naphthyl, heteroaryl, and (CH₂)ₙ are unsubstituted or substituted with one to three groups independently selected from R³;
      each R³ is independently selected from the group consisting of
(1) C₁₋₆ alkyl,
(2) —(CH₂)ₙ-phenyl,
(3) —(CH₂)ₙ-naphthyl,
(4) —(CH₂)ₙ-heteroaryl,
(5) —(CH₂)ₙ-heterocycloalkyl,
(6) —(CH₂)ₙC₃₋₇ cycloalkyl,
(7) halogen,
(8) OR⁴,
(9) —(CH₂)ₙN(R⁴)₂,
(10) NO₂,
(11) —(CH₂)ₙNR⁴SO₂R⁴,
(12) —(CH₂)ₙSO₂N(R⁴)₂,
(13) —(CH₂)ₙS(O)ₚR⁴,
(14) CF₃,
(15) CH₂CF₃,
(16) OCF₃, and
(17) OCH₂CF₃;
   in which heteroaryl is as defined above; alkyl, phenyl, naphthyl, heteroaryl, cycloalkyl, and heterocycloalkyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
   each R⁴ is independently selected from the group consisting of
(1) hydrogen,
(2) C₁₋₆ alkyl,
(3) —(CH₂)ₙ-phenyl,
(4) —(CH₂)ₙ-heteroaryl,
(5) —(CH₂)ₙ-naphthyl,
(6) —(CH₂)ₙ-heterocycloalkyl,
(7) —(CH₂)ₙC₃₋₇ cycloalkyl, and
(8) —(CH₂)ₙC₃₋₇ bicycloalkyl;
   wherein alkyl, phenyl, heteroaryl, heterocycloalkyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl; and
   n is 0, 1, 2, 3 or 4;
   comprising the steps of:
   (a) hydrolyzing a pyrrolidine compound of structural formula (X), wherein Y, R¹ and R² are as defined above, 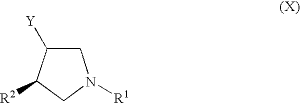
   with an aqueous base in a solvent; and
   (b) isolating the resulting product.

In another embodiment of the present invention, the pyrrolidine compound of formula (X) is hydrolyzed with a base selected from the group consisting of NaOH, LiOH and KOH. In one class of this embodiment, the base is NaOH. In a subclass of this class, the base is aqueous NaOH.

In another embodiment of the present invention, the pyrrolidine compound of formula (X) is hydrolyzed in a solvent selected from the group consisting of methanol, ethanol, and isopropanol. In a class of this embodiment, the solvent is ethanol.

The present invention also provides a process for the preparation of compounds of structural formula (XIX): 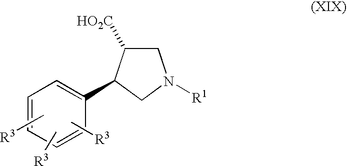
wherein
R¹ is selected from the group consisting of
(1) hydrogen,
(2) amidino,
(3) C₁₋₄ alkyliminoyl,
(4) C₁₋₁₀ alkyl,
(5) —(CH₂)ₙ—C₃₋₇ cycloalkyl,
(6) —(CH₂)ₙ-phenyl,
(7) —(CH₂)ₙ-naphthyl, and
(8) —(CH₂)ₙ-heteroaryl,
   in which phenyl, naphthyl, and heteroaryl are unsubstituted or substituted with one to three groups independently selected from R³; and alkyl, cycloalkyl, and (CH₂)ₙ are unsubstituted or substituted with one to three groups independently selected from R³ and oxo; each R³ is independently selected from the group consisting of
(1) C₁₋₆ alkyl,
(2) —(CH₂)ₙ-phenyl,
(3) —(CH₂)ₙ-naphthyl,
(4) —(CH₂)ₙ-heteroaryl,
(5) —(CH₂)ₙ-heterocycloalkyl,
(6) —(CH₂)ₙC₃₋₇ cycloalkyl,
(7) halogen,
(8) OR⁴,
(9) —(CH₂)ₙN(R⁴)₂,
(10) NO₂,
(11) —(CH₂)ₙNR⁴SO₂R⁴,
(12) —(CH₂)ₙSO₂N(R⁴)₂,
(13) —(CH₂)ₙS(O)ₚR⁴,
(14) CF₃,
(15) CH₂CF₃,
(16) OCF₃, and
(17) OCH₂CF₃;
   in which heteroaryl is as defined above; alkyl, phenyl, naphthyl, heteroaryl, cycloalkyl, and heterocycloalkyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group; each R⁴ is independently selected from the group consisting of
(1) hydrogen,
(2) C₁₋₆ alkyl,
(3) —(CH₂)ₙ-phenyl,
(4) —(CH₂)ₙ-heteroaryl,
(5) —(CH₂)ₙ-naphthyl,
(6) —(CH₂)ₙ-heterocycloalkyl,
(7) —(CH₂)ₙC₃₋₇ cycloalkyl, and
(8) —(CH₂)ₙC₃₋₇ bicycloalkyl;
   wherein alkyl, phenyl, heteroaryl, heterocycloalkyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl; and
   n is 0, 1, 2, 3, or 4;
   comprising the steps of:
(a) preparing an alcohol of structural formula (XIII) 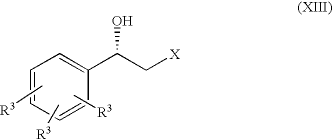
   wherein X is bromide or chloride, and R³ is as defined above,
   by treating a ketone of structural formula (XII), 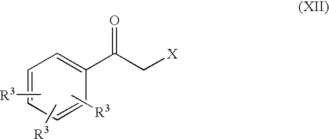
   wherein X is bromide or chloride, and R³ is as defined above, with a reducing agent, and isolating the resulting product;
(b) forming an amino alcohol of structural formula (XV) 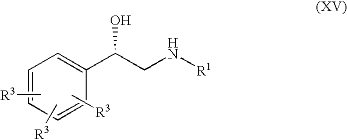
   wherein R¹ and R³ are as defined above,
   by treating an alcohol of structural formula (XIII) 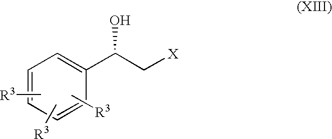
   wherein X is chloride or bromide and R³ are as defined above,
   with an amine of general formula R¹NH₂, wherein R¹ is as defined above, and a base in a solvent, and isolating the resulting product;
(c) forming a compound of structural formula (XVI), wherein Y is —CN or —CO₂R⁵ and R⁵ is C₁₋₄ alkyl, and R¹ and R³ are as defined above, 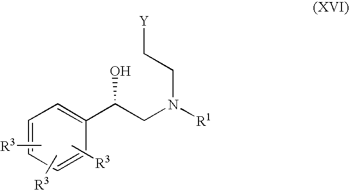
   by treating the amino alcohol of structural formula (XV) wherein R¹ and R³ are as defined above, 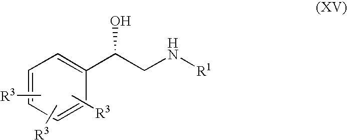
   with a compound of general formula (XI) 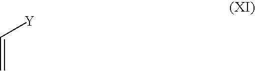
   wherein Y is —CN or —CO₂R⁵, and R⁵ is C₁₋₄ alkyl, and isolating the resulting product;
(d) forming a pyrrolidine compound of structural formula (XVIII) wherein Y, R¹ and R³ are as defined above, 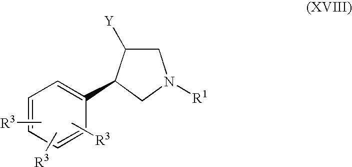
   by treating the compound of structural formula (XVI), wherein Y, R¹ and R³ are as defined above, 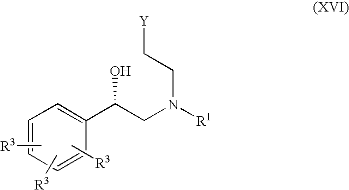
   with an alcohol activating reagent, followed by a base;
(e) forming a pyrrolidine acid of structural formula (XIX), wherein R¹ and R³ are as defined above, 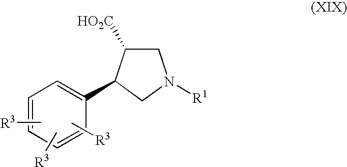
   by hydrolyzing the pyrrolidine compound of structural formula (XVIII), wherein Y, R¹ and R³ are as defined above, 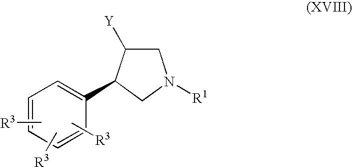
   with an aqueous base in a solvent; and
(f) isolating the resulting product.

In one embodiment, R³ is selected from the group consisting of halogen, —CF₃, and OR⁴. In a class of this embodiment of the present invention, R³ is selected from the group consisting of fluoride, bromide, chloride, —CF₃, and —OC₁₋₆ alkyl. In a subclass of this class, R³ is selected from fluoride, bromide, CF₃, and —OCH₃.

In another embodiment of the present invention, the reducing agent used to treat the compound of formula (XII) of step (a) is (+)-DIP chloride.

In another embodiment of the present invention, the compound of formula (XII) of step (a) is treated with a reducing agent in the presence of a catalyst. In a class of this embodiment the reducing agent is selected from the group consisting of borane-N,N-diethyl aniline, borane-THP, and borane-dimethylsulfide. In a subclass of this class, the reducing agent is borane-N,N-diethyl aniline. In another class of this embodiment, the catalyst is selected from the group consisting of (S)-CBS and (S)-2-methyl CBS oxazaborolidine. In a subclass of this class, the catalyst is (S)-2-methyl CBS oxazaborolidine.

In another embodiment of the present invention, alcohol of formula (XIII) is treated with an amine of general formula R¹NH₂, wherein R¹ is selected from the group consisting of hydrogen, —(CH₂)ₙphenyl, or C₁₋₆alkyl. In a class of this embodiment, R¹ is tert-butyl or —CH₂-phenyl. In a subclass of this class, R¹ is tert-butyl.

In another embodiment of the present invention, the alcohol of formula (XIII) is treated with a base selected from the group consisting of NaOH, LiOH, KOH. In a class of this embodiment, the base is NaOH.

In another embodiment of the present invention, the alcohol of formula (XIII) is treated in a solvent selected from methanol or ethanol. In a class of this embodiment, the solvent is methanol. In a subclass of this class, the solvent is refluxing methanol.

In another embodiment of the present invention, the amino alcohol of structural formula (XV) is isolated by recrystallization from heptane or hexane. In a class of this embodiment, the solvent is heptane.

In another embodiment of the present invention, the compound of formula (XI) is the compound wherein Y is CN.

In another embodiment of the present invention, the compound of formula (XI) is the compound wherein Y is —CO₂R⁵, wherein R⁵ is C₁₋₄ alkyl. In a class of this embodiment Y is —CO₂CH₃, —CO₂CH₂CH₃, or —CO₂CH₂CH₂CH₂CH₃. In a subclass of this class, Y is —CO₂CH₂CH₃, or —CO₂CH₂CH₂CH₂CH₃.

In another embodiment of the present invention, the compound of structural formula (XVI) is formed by heating the mixture to reflux.

In another embodiment of the present invention, the compound of structural formula (XVI) is formed by adding ethanol, formamide or a mixture thereof. In a class of this embodiment, the compound of structural formula (XVI) is formed by adding a 1:1 mixture of ethanol:formamide.

In another embodiment of the present invention, the compound of structural formula (XVI is isolated by recrystallizing from heptane or hexane.

In another embodiment of the present invention, the compound of structural formula (XVI) is treated with an alcohol activating reagent selected from the group consisting of ClPO(OR⁶)₂, ClPO(N(R⁶)₂)₂, MsCl, Ms₂O, TsCl, and Ts₂O, wherein R⁶ is C₁₋₄ alkyl or phenyl. In a class of this embodiment, the alcohol activating reagent is chlorodiethyl phosphate.

In another embodiment of the present invention, the compound of structural formula (XVI) is treated with a base selected from the group consisting of lithium hexamethyldisilazide, sodium hexamethyl disilazide, and potassium hexamethyldisilazide. In a class of this embodiment, the base is lithium hexamethyl disilazide.

In another embodiment of the present invention, the compound of structural formula (XVI) is treated at a temperature of about −30 to about +10 C. In a class of this embodiment, the temperature is about −15 C.

In another embodiment of the present invention, the pyrrolidine compound of formula (XVIII) is hydrolyzed with a base selected from the group consisting of NaOH, LiOH and KOH. In one class of this embodiment, the base is NaOH. In a subclass of this class, the base is aqueous NaOH.

In another embodiment of the present invention, the pyrrolidine compound of formula (XVI) is hydrolyzed in a solvent selected from the group consisting of methanol, ethanol, and isopropanol. In a class of this embodiment, the solvent is ethanol.

In another embodiment, the product of step (f) is isolated by forming a zwitterion of the trans pyrrolidine acid of structural formula (XX) 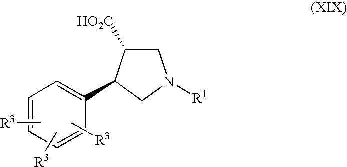
wherein R¹ and R³ are as defined above; recrystallizing the zwitterion from a solvent; and isolating the resulting product.

In a class of this embodiment the zwitterion of the pyrrolidine acid of formula (XIX) is formed at the isoelectric pH using an acid. In one subclass of this class, the acid is selected from sulfuric acid or hydrochloric acid. In a subclass of this subclass, the acid is sulfuric acid. In another subclass of this class, the isoelectric pH is about 6 and a stoichiometric amount of acid is added.

In another class of this embodiment, the zwitterion of the pyrrolidine acid of formula (XIX) is recrystallized from a solvent selected from the group consisting of ethanol, isopropyl alcohol, methyl tert-butyl ether or a mixture thereof. In a subclass of this class, the solvent is a mixture of isopropyl alcohol and methyl tert-butyl ether. In a subclass of this subclass, the solvent is 1:3 isopropyl alcohol:methyl tert-butyl ether.

The present invention also provides a process for the preparation of compounds of structural formula (XIX): 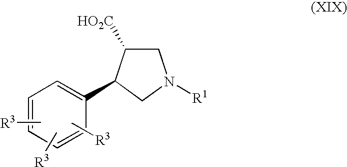
wherein
R¹ is selected from the group consisting of
(1) hydrogen,
(2) amidino,
(3) C₁₋₄ alkyliminoyl,
(4) C₁₋₁₀ alkyl,
(5) —(CH₂)ₙ—C₃₋₇ cycloalkyl,
(6) —(CH₂)ₙ-phenyl,
(7) —(CH₂)ₙ-naphthyl, and
(8) —(CH₂)ₙ-heteroaryl,
   in which phenyl, naphthyl, and heteroaryl are unsubstituted or substituted with one to three groups independently selected from R³; and alkyl, cycloalkyl, and (CH₂)ₙ are unsubstituted or substituted with one to three groups independently selected from R³ and oxo; each R³ is independently selected from the group consisting of
(1) C₁₋₆ alkyl,
(2) —(CH₂)ₙ-phenyl,
(3) —(CH₂)ₙ-naphthyl,
(4) —(CH₂)ₙ-heteroaryl,
(5) —(CH₂)ₙ-heterocycloalkyl,
(6) —(CH₂)ₙC₃₋₇ cycloalkyl,
(7) halogen,
(8) OR⁴,
(9) —(CH₂)ₙN(R⁴)₂,
(10) NO₂,
(11) —(CH₂)ₙNR⁴SO₂R⁴,
(12) —(CH₂)ₙSO₂N(R⁴)₂,
(13) —(CH₂)ₙS(O)ₚR⁴,
(14) CF₃,
(15) CH₂CF₃,
(16) OCF₃, and
(17) OCH₂CF₃;
   in which heteroaryl is as defined above; alkyl, phenyl, naphthyl, heteroaryl, cycloalkyl, and heterocycloalkyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
   each R⁴ is independently selected from the group consisting of
(1) hydrogen,
(2) C₁₋₆ alkyl,
(3) —(CH₂)ₙ-phenyl,
(4) —(CH₂)ₙ-heteroaryl,
(5) —(CH₂)ₙ-naphthyl,
(6) —(CH₂)ₙ-heterocycloalkyl,
(7) —(CH₂)ₙC₃₋₇ cycloalkyl, and
(8) —(CH₂)ₙC₃₋₇ bicycloalkyl;
   wherein alkyl, phenyl, heteroaryl, heterocycloalkyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl; and
   n is 0, 1, 2, 3 or 4;
   comprising the steps of:
   (a) hydrolyzing a pyrrolidine compound of structural formula (XVIII), wherein Y, R¹ and R³ are as defined above, 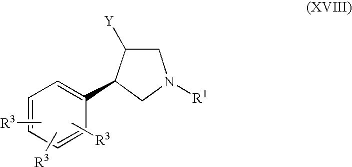
   with an aqueous base in a solvent; and
   (b) isolating the resulting product.

In another embodiment of the present invention, the pyrrolidine compound of formula (XVIII) is hydrolyzed with a base selected from the group consisting of NaOH, LiOH and KOH. In one class of this embodiment, the base is NaOH. In a subclass of this class, the base is aqueous NaOH.

In another embodiment of the present invention, the pyrrolidine compound of formula (VIII) is hydrolyzed in a solvent selected from the group consisting of methanol, ethanol, and isopropanol. In a class of this embodiment, the solvent is ethanol.

In a further embodiment of this invention, the compound of formula I is compound 1-9 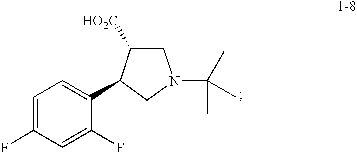
or a zwitterion or salt thereof. In a class of this embodiment, the zwitterion is formed by the addition of sulfuric acid or hydrochloric acid. In another class of this embodiment, the zwitterion is formed by the addition of sulfuric acid.

In a further embodiment of this invention, the compound of formula I is compound 2 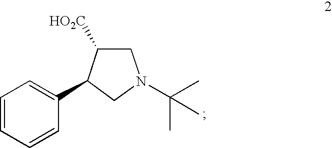
or a zwitterion or salt thereof. In a class of this embodiment, the zwitterion is formed by the addition of sulfuric acid or hydrochloric acid. In another class of this embodiment, the zwitterion is formed by the addition of sulfuric acid.

In a further embodiment of this invention, the compound of formula I is compound 3 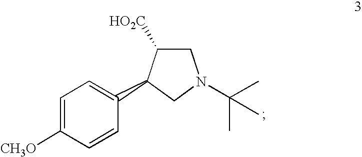
or a zwitterion or salt thereof. In a class of this embodiment, the zwitterion is formed by the addition of sulfuric acid or hydrochloric acid. In another class of this embodiment, the zwitterion is formed by the addition of sulfuric acid.

Throughout the instant application, the following terms have the indicated meanings:

The alkyl groups specified above are intended to include those alkyl groups of the designated length in either a straight or branched configuration. Exemplary of such alkyl groups are methyl, ethyl, propyl, isopropyl, butyl, sec-butyl, tertiary butyl, pentyl, isopentyl, hexyl, isohexyl, and the like.

The term “halogen” is intended to include the halogen atoms fluorine, chlorine, bromine and iodine.

The term “aryl” includes phenyl and naphthyl.

The term “heteroaryl” includes mono- and bicyclic aromatic rings containing from 1 to 4 heteroatoms selected from nitrogen, oxygen and sulfur. “5- or 6-Membered heteroaryl” represents a monocyclic heteroaromatic ring. Examples of heteroaryls useful in this invention include wherein heteroaryl is selected from the group consisting of pyridinyl, furyl, thienyl, pyrrolyl, oxazolyl, thiazolyl, imidazolyl, pyrazolyl, isoxazolyl, isothiazolyl, pyrimidinyl, pyrazinyl, pyridazinyl, quinolyl, isoquinolyl, benzimidazolyl, benzofuryl, benzothienyl, indolyl, benzthiazolyl, and benzoxazolyl, and the like. Bicyclic heteroaromatic rings include, but are not limited to, benzothiadiazole, indole, benzothiophene, benzofuran, benzimidazole, benzisoxazole, benzothiazole, quinoline, benzotriazole, benzoxazole, isoquinoline, purine, furopyridine and thienopyridine. In one embodiment of the present invention, heteroaryl is selected from the group consisting of pyridinyl, furyl, thienyl, pyrrolyl, oxazolyl, thiazolyl, triazolyl, triazinyl, tetrazolyl, thiadiazolyl, imidazolyl, pyrazolyl, isoxazolyl, isothiazolyl, oxathiazolyl, pyrimidinyl, pyrazinyl, pyridazinyl, quinolyl, isoquinolyl, benzimidazolyl, benzofuryl, benzothienyl, indolyl, benzthiazolyl, and benzoxazolyl.

The term “cycloalkyl” is intended to include non-aromatic rings containing only carbon atoms such as cyclopropyl, cyclobutyl, cyclopentyl, cyclohexyl, and cycloheptyl.

The term “heterocycloalkyl” is intended to include non-aromatic heterocycles containing one to four heteroatoms selected from nitrogen, oxygen and sulfur. Examples of a 5 or 6-membered heterocycloalkyl include piperidine, morpholine, thiamorpholine, pyrrolidine, imidazolidine, tetrahydrofuran, piperazine, and the like.

Certain of the above defined terms may occur more than once in the above formula and upon such occurrence each term shall be defined independently of the other; thus for example, NR⁴R⁴ may represent NH₂, NHCH₃, N(CH₃)CH₂CH₃, and the like.

The process and intermediates of the present invention can be exemplified with the preparation of (3S,4R)-N-tert-Butyl-4(2,4-difluorophenyl)-pyrrolidine 3-carboxylic acid (1-9) as shown in Scheme 1.

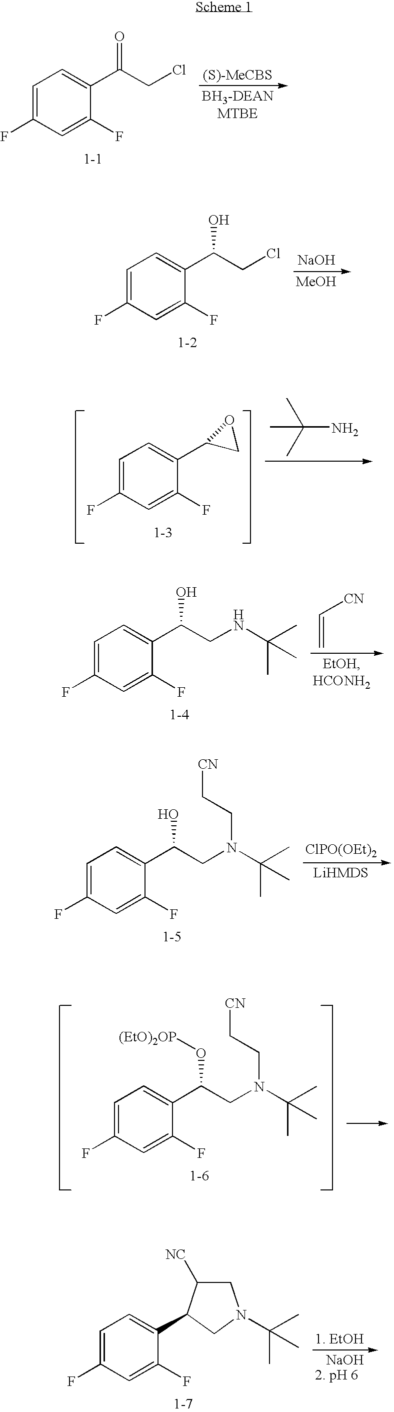

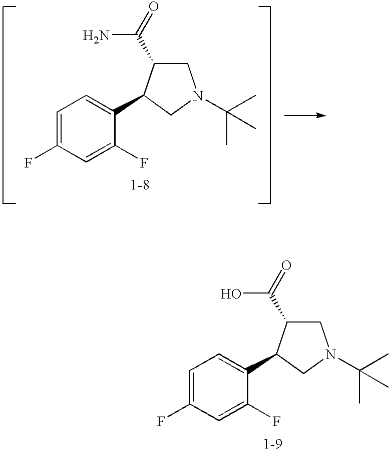

As shown in Scheme 1, the known (3S,4R)-N-tert-Butyl-4-(2,4-difluorophenyl)pyrrolidine 3-carboxylic acid (1-9) is prepared as follows.

The asymmetric reduction of 2-chloro-2′,4′-difluoroacetophenone 1-1 with a reducing agent, such as (+) DIP chloride; or with a reducing agent such as borane-diethyl aniline, borane dimethyl-sulfide, or borane-THF in the presence of a catalyst, such as (S)CBS, or (S)-2-methyl CBS oxazaborolidine. The reaction is run in a solvent such as diisopropyl ether, MTBE, toluene, or THF, at a temperature of about −20 to +60° C., and optimally at a temperature of about +30 to +50° C., to afford the (S)-alcohol 1-2. When (S)-2-methyl CBS oxazaborolidine and borane-diethyl aniline are used for the reduction, and the reduction is run at a temperature of about 40° C., then the use of 0.5 mole % of (S)-CBS catalyst results in the formation of 98.88% ee of the S-enantiomer of alcohol 1-2. The R-enantiomer of alcohol 1-2 may be prepared by treating 1-1 with (−) DIP chloride, or by treating 1-1 with a borane reducing agent and a catalyst, such as (R)-CBS or (R)-2-methyl CBS oxazaborolidine under similar reaction conditions. By reducing 1-1 with the (−) DIP chloride, or with a borane reducing agent and (R)-CBS or (R)-2-methyl CBS oxazaborolidine, the 3R,4S diastereomer of 1-1 may be made in a similar fashion. The reduction of acetophenone 1-1 may also be affected by treatment with sodium borohydride and trimethylsilyl chloride catalyzed by (S)-α,α-diphenyl pyrrolidine methanol, or by treatment of acetophenone 1-1 via asymmetric transfer hydrogenation using chiral rhodium complex catalysis.

Treatment of alcohol 1-2 with a base, such as sodium hydroxide, lithium hydroxide or potassium hydroxide, in a protic solvent, such as methanol or ethanol, and subsequently heating to reflux results in the formation of the epoxide intermediate 1-3 in situ. Opening the epoxide ring with a primary amine, such as a C₁₋₆ alkyl amine, benzyl amine or substituted benzylamine, affords the amino alcohol 1-4. Crystallization of 1-4 from heptane or hexanes gives amino alcohol 1-4 as >99.9% ee of the S-enantiomer. When methanol and tert-butyl amine are used to prepare amino alcohol 1-4, the optimal ratio of methanol to tert-butyl amine is 1:5. The treatment of the epoxide intermediate 1-3 with benzyl amine and the subsequent removal of the benzyl protecting group under standard conditions, such as hydrogenation, is useful to prepare compounds of formula I in which R¹ is H.

Treatment of amino alcohol 1-4 with acrylonitrile and heating to reflux, followed by the addition of ethanol, formamide, or a mixture thereof, in the later stages of the reaction, affords the amino nitrile 1-5. The amino nitrile 1-5 may be further purified by recrystallizing from heptane or hexane.

The pyrrolidine nitrile 1-7 was formed by the conversion of the alcohol of nitrile 1-5 into a leaving group by treatment with an alcohol activating reagent, such as ClPO(OEt)₂, to form intermediate 1-6 in situ. Subsequent treatment of intermediate 1-6 with a base, such as lithium hexamethyldisilazide, sodium hexamethyldisilazide or potassium hexamethyldisilazide, at a temperature of about −30 to about +10° C. yields a cis/trans mixture of the pyrrolidine nitrile 1-7. Other alcohol activating reagents useful to convert the alcohol into a leaving group include, but are not limited to, ClPO(OR⁶)₂, ClPO(N(R⁶)₂)₂, MsCl, Ms₂O, TsCl or Ts₂O, wherein R⁶ is C₁₋₄alkyl or phenyl.

Acid 1-9 is formed from nitrile 1-7 via the amide intermediate 1-8. The kinetically controlled hydrolysis/epimerization of pyrrolidine nitrile 1-7 with an aqueous base, such as sodium hydroxide, lithium hydroxide or potassium hydroxide, in a protic solvent, such as methanol, ethanol, or isopropanol, at reflux, and the subsequent adjustment of the pH to the isoelectric point of 1-9 with an acid, such as sulfuric acid or HCl, affords the zwitterion of 1-9. The pH at the isoelectric point is about pH 6. The zwitterion of 1-9 may be recrystallized from ethanol to give the trans pyrrolidine acid zwitterion of 1-9. The zwitterion of 1-9 may also be recrystallized as an HCl salt from acetonitrile.

Abbreviations Used in the Description of the Preparation of the Compounds of the Present Invention: (S)-Me CBS and (S)-2-methyl-CBS-OAB are (S)-2-methyl CBS oxazaborolidine; BOC is tert-butyl carbamate; DEAN is diethyl aniline; DMF is N,N-dimethyl formamide; EtOAc is ethyl acetate; EtOH is ethanol; g is grams; h or hr is hours; H₂ is hydrogen; HCl is hydrochloric acid, HPLC is high pressure liquid chromatography; mm Hg is millimeters of mercury; IPA is isopropyl alcohol; kg is kilograms; L is liters; LiHMDS is lithium hexamethyl disilazide; M is molar; mL is milliliters; MeOH is methanol, min is minutes, mol is moles; Ms is methanesulfonyl; MTBE is methyl t-butyl ether; N is normal; NMP is N-methylpyrrolidinone; NaCl is sodium chloride; NMR is nuclear magnetic resonance; OAc is acetate; Ts is toluenesulfonyl; THF is tetrahydrofuran; and ClPO(OEt)₂ is chloro diethyl phosphate.

The following Example is provided to illustrate the invention and is not to be construed as limiting the scope of the invention in any manner. A representative experimental procedure utilizing the novel process is detailed below. For purposes of illustration, the following Example is directed to the preparation of compound 1-9 but doing so is not intended to limit the present invention to a process for making that specific compound.

### EXAMPLE 1

### (3S,4R)-N-tert-Butyl-4(2,4-difluorophenyl)pyrrolidine 3-carboxylic acid (1-9)

Step A:

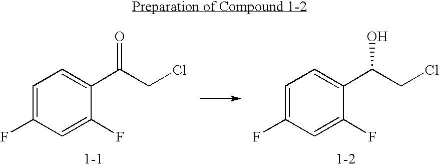

A solution of (S)-2-methyl-CBS-OAB (128 mL of 1.0M solution in toluene, Aldrich), borane-N,N-diethylaniline (25.7 mol, Callery) in MTBE (10 L) was heated to 38-42° C., followed by the addition of a solution of 2-chloro-2′,4′-difluoro-acetophenone (4891 g, Apollo) in MTBE (14.7 L) over 10 hours. The resulting homogeneous solution was stirred at 40° C. for one hour, and then cooled to 18° C. and stirred overnight. Methanol (2.3 L) was added over 60 minutes, while maintaining the temperature at <20° C. with cooling. The resulting homogeneous solution was stirred for 30 minutes, then dilute with water (24 L) and 5 N aqueous HCl (10 L) was added over 30 minutes, while maintaining the temperature at 22-25° C. with cooling. After stirring 30 minutes, the layers were separated. The organic layer was washed with saturated aqueous NaCl, and then concentrated in vacuo to give chloro-alcohol 1-2. The chiral assay of the chloro-alcohol gave a 99.44:0.56 ratio of S:R enantiomers (98.88% ee).

¹H-NMR (CDCl₃, 400.25 MHz) δ 7.51 (m, 1H), 6.91 (m, 1H), 6.80 (m, 1H), 5.16 (dd, J=8.2, 3.2 Hz, 1H), 3.79 (dd, J=11.2, 3.4 Hz, 1H), 3.62 (dd, J=11.2, 8.2 Hz, 1H), 3.02 (s, 1H).

¹³C NMR (CDCl₃, 100.65 MHz) δ 162.7 (dd, J=249.6, 12.0 Hz), 159.7 (dd, J=248.5, 11.7 Hz), 128.6 (dd, J=9.7, 5.7 Hz), 123.0 (dd, J=13.5, 3.8 Hz), 111.6 (dd, J=21.2, 3.7 Hz), 103.8 (t, J=25.4 Hz), 67.8 (d, 2.1 Hz), 49.4.

BP: 69-71° C. at 15 mm Hg.
Step B:

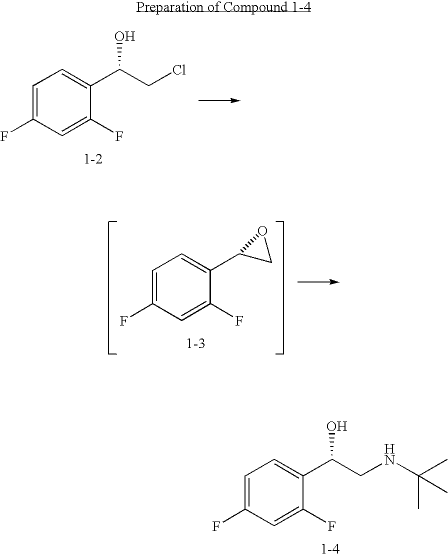

The concentrated MTBE solution of 1-2 from Step A (5040 g, 25.67 mol) was diluted with methanol (5 L), then tert-butylamine (25 L) was added. The mixture warmed upon mixing to 45° C. The mixture was then cooled to 25° C. and solid NaOH pellets (1048 g) were added. No exotherm was observed, and the mixture was stirred and warmed to reflux. After 2 hours, if chloro-alcohol remains, additional NaOH can be added. After 12-20 hours of refluxing, the mixture was concentrated in vacuo to 1/3 volume, then water (5 L) and MTBE (20 L) were added. The resulting layers were separated, and the aqueous phase was re-extracted with MTBE (2×2 L). The combined extracts were washed with saturated aqueous NaCl (1 L), then concentrated in vacuo. Heptane (40 L) was added and the concentration was continued to bring the volume to 20 L. The resulting mixture was then heated to −90° C. to dissolve all solids, and allowed to cool to 22° C. to crystallize over 4 hours. The mixture was then cooled to 0° C., stirred 12-15 hr, and filtered. The filtrate was washed with cold heptane (2×5 L), then dried in vacuo at 35° C. to obtain the crystalline amino-alcohol 1-4. The chiral assay of 1-4 gave a >99.95:0.05 ratio of S:R enantiomers (>99.9% ee).

¹H-NMR (CDCl₃, 400.25 MHz) δ 7.52 (m, 1H), 6.88 (n, 1H), 6.76 (m, 1H), 4.85 (dd, J=8.6, 3.4, 1H), 2.94 (m, 1H), 2.52 (m, 1H), 1.10 (s, 9H).

¹³C NMR (CDCl₃, 100.65 MHz) δ 162.1 (dd, J=247.4, 12.0), 159.7 (dd, J=247.9, 12.0), 128.3 (dd, J=13.6, 3.8), 111.1 (dd, J=20.9, 3.5), 103.4 (t, J=32.0), 66.0, 50.4, 48.7, 29.1 (3C). MP (DSC): onset 115.35° C., end 118.66° C., peak 117.22° C.

Anal. Calcd for C₁₂H₁₇F₂NO: Calc., C, 62.87, H, 7.47, F, 16.57, N, 6.11. Found, C, 62.93, H, 7.67, F, 16.24, N, 6.13.
Step C:

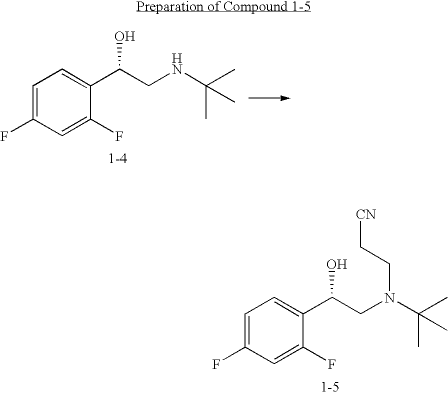

A mixture of aminoethanol 1-4 from Step B (5.205 kg, 22.68 mol) and acrylonitrile (26.9 L, 408 mol) was heated at reflux (−77° C.) under a nitrogen atmosphere. After heating for 20 hours (with ˜90% conversion), one equivalent each of ethanol (1.32 L, 22.68 mol) and formamide (0.9 L, 22.68 mol) was added, and heating was continued for 12 hours. After cooling to 22° C., the solution was concentrated by distillation (80-90 torr at 20-22° C. pot temperature) to 12 L volume. The resulting residue was diluted with isopropyl acetate (22 L) and re-concentrated (55-75 torr and 22-27° C. pot temperature). The dilution and re-concentration was repeated, and then the resulting residue was diluted with isopropyl acetate to a total volume of 34 L. Gummy polymer that was present was allowed to settle after stopping the stirrer, and the bulk of the supernatant was filtered (10-15 um porosity), followed by the rest of material. The filter cake was washed with isopropyl acetate and the filtrate was diluted with a total of 24 L of isopropyl acetate. The combined filtrate (˜54 L) was washed with a solution made up of water (31.2 L), acetic acid (52 mL, 4 mol %), and saturated brine (3.1 L). This was followed by a 12% aqueous NaCl wash (2×34 L). The organic layer was concentrated (15-45 torr and 5-29° C.) to −15 L volume and flushed with 5×6 L portions of n-heptane, during which time product crystallized. The slurry was diluted with n-heptane to a volume of 23 L. The mixture was stirred at 0-5° C. for 3 days, then filtered and washed with cold (5° C.) n-heptane (14 L). The wet cake was dried in vacuo at 20° C. with a nitrogen sweep for 4 days to afford nitrile 1-5 as a crystalline white solid. The chiral assay of crystalline nitrile 1-5 was >99.99 area % as the desired S-enantiomers.

¹H-NMR (400.25 MHz, CDCl₃) δ 7.55 (m, 1H), 6.90 (m, 1H), 6.77 (m, 1H), 4.84 (dd, J=10.2, 3.1, 1H), 3.66 (OH, 1H), 3.00-2.83 (om, 3H), 2.62-2.47 (om, 2H), 2.45 (dd, J=13.9, 10.3, 1H), 1.15 (s, 9H).

¹³C-NMR (100.65 MHz, CDCl₃) δ 162.1 (dd, J=247.7, 11.9), 159.6 (dd, J=247.5, 11.9), 128.0 (dd, J=9.5, 6.5), 125.1 (dd, 13.7, 3.6), 118.6, 111.4 (dd, J=20.9, 3.3), 103.4 (t, J=25.6), 65.4, 57.9, 55.7, 47.3, 27.2 (3C), 20.2.

¹⁹F-NMR (376.61 MHz, CDCl₃) δ−112.25 (d, J=6.9), −116.27 (d, 6.8).

MP (DSC): onset 60.20° C., end 64.15° C., peak 62.61° C.

Anal. Calcd for C₁₅H₂₀F₂N₂O: Calc., C, 63.81, H, 7.14, N, 9.92, F, 13.46. Found, C, 63.79, H, 7.30, N, 9.93, F, 13.31.
Step D:

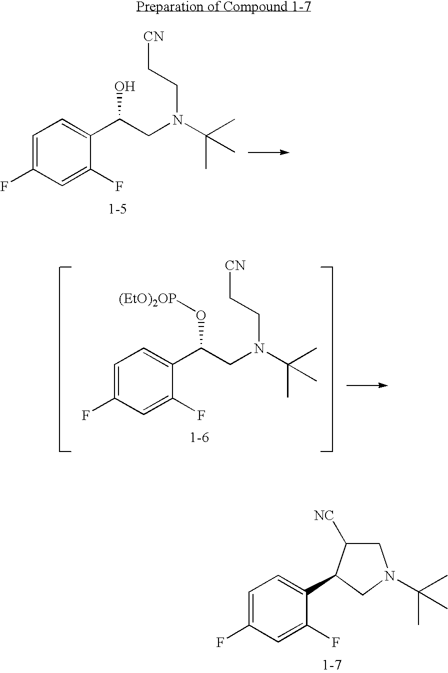

To a solution of alcohol 1-5 (5.73 kg, 99.9%, 20.28 mol) in dry THP (31.3 L), cooled to −20° C., was added chloro diethylphosphate (3.79 kg, 21.29 mol). Lithium hexamethyldisilazide (1.35 M in THF; 31.5 L, 42.58 mol) was slowly added over 1.5 hours while maintaining the reaction temperature at −15±3° C. After stirring at −15° C. for 2 hours, the HPLC assay confirmed complete conversion to pyrrolidine 1-7 (as a 80:20 trans:cis mixture).

The reaction mixture was quenched with water (50.6 L) at <15° C. and extracted with n-heptane (40.5 L) at 20° C. The organic layer was washed with 10% aqueous NaCl solution (52 L). The organic layer was carefully extracted with 3 N HCl solution (40.6 L, 121.8 mol) with cooling to keep the temperature <35° C. The aqueous layer (58 L) was adjusted to pH 11-12 with 50% aq NaOH (6.13 L, 116.1 mol) and extracted with n-heptane (54 L). The layers were separated. The organic layer was washed once with 10% aqueous NaCl solution (26 L) and the resulting heptane solution (48 kg total) was assayed by HPLC to contain cyclized nitrile 1-7 (as a 80:20 trans:cis mixture), which was used, as is, in the hydrolysis/epimerization reaction in Step E.

Trans-Pyrrolidine Nitrile-HCl Salt

¹H-NMR (400.25 MHz, D₂O) δ 7.42 (m, 1H), 7.03-6.96 (om, 2H), 4.06-3.79 (om, 5H), 3.46 (bt, J=11.6, 1H), 1.38 (s, 9H).

¹³C-NMR (100.65 MHz, D₂O) δ 163.2 (dd, J=180.9, 12.6), 160.8 (dd, J=180.8, 12.7), 130.2 (dd, J=10.2, 5.4), 116.9, 116.8, 112.1 (dd, J=21.7, 3.4), 104.6 (t, J=26.0), 63.2, 51.1, 49.3, 41.4, 32.3, 23.7 (3C).

¹⁹F-NMR (376.61 MHz, D₂O) δ−109.87 (d, J=7.7), −112.87 (d, J=8.5).

MP (DSC): onset 179.23° C., end 182.83° C., peak 181.85° C.

HR-MS M+H theoretical 265.1516; found 265.1517.

Cis-Pyrrolidine Nitrile-HCl Salt

¹H-NMR (d₄-CH₃OH, 400.25 MHz) δ 7.57 (ma, 1H), 7.16-7.03 (om, 2H), 4.82 (s, OH), 4.20-4.08 (m, 2H), 4.07-3.90 (m, 3H), 3.89-3.76 (m, 1H), 1.53 (s, 9H).

¹³C-NMR (d₄-CH₃OH, 100.65 MHz) δ 165.0 (dd, J=193.3, 12.5), 162.5 (dd, J=192.9, 12.5), 131.5, 118.9 (dd, J=14.3, 3.7), 118.3, 113.0 (dd, J=21.7, 3.5), 105.4 (t, J=26.2), 64.2, 51.8, 51.1, 40.2, 35.0, 24.9 (3C)

¹⁹F-NMR (376.61 MHz, d₄-CH₃OH) δ−111.29, −112.61 (d, J=6.8).

MP (DSC): onset 257.91° C., end 263.37° C., peak 262.15° C.

Anal. Calcd for C₁₅H₁₉ClF₂N₂: Calc., C, 59.90, H, 6.37, N, 9.31, F, 12.63, Cl, 11.79. Found, C, 59.76, H, 6.26, N, 9.40, F, 12.54, Cl, 11.43.
Step E:

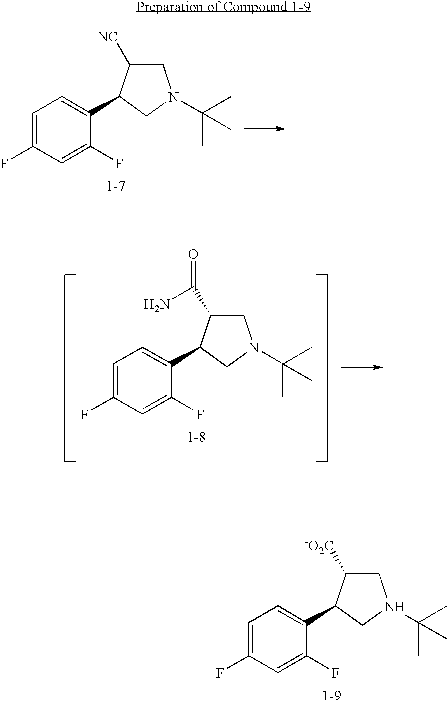

A solution of crude pyrrolidine nitrile 1-7 (4.88 kg, 18.46 mol) in n-heptane (˜65 L total) from Step D was solvent-switched to ethanol (˜20.6 L total) by distilling the n-heptane (50-60 torr, 25° C.) down to about 6 L in volume, and adding ethanol (15 L). The resulting solution was concentrated to a 6 L volume, and diluted with ethanol (14.6 L) to give a total volume of 20.6 L. To this solution was added 50% aqueous NaOH (2.7 L, 51.15 mol) over 2 minutes with stirring. This mixture was then heated to reflux (78-80° C.) under nitrogen for 5 to 6 hours. The reaction was monitored by HPLC. After cooling to 20° C., the reaction mixture was diluted with ethanol (25.4 L) and methanol (40.6 L) to give a total volume of −88 L (as a 1:1 MeOH:EtOH mixture). This solution was cooled to 12° C. and 96% H₂SO₄ (1.42 L, 25.6 mol) was added, while maintaining the temperature at about 20° C. The slurry was filtered through a bed of Solka-Floc (5 kg) and anhydrous powder Na₂SO₄ (4 kg), and then washed with 1:1 EtOH:MeOH (60 L). The resulting filtrate was re-filtered, concentrated and solvent-switched to a 2-propanol solution (−15 L volume) by vacuum-distillation. The product crystallized during solvent switching.

The resulting slurry was heated to reflux (−80° C.) for 2 hours (which only partly dissolves product). The mixture was then allowed to cool. After cooling to 16° C., MTBE (30.4 L, 3 volumes relative to IPA) was added to the mixture over 5 hours to give a 1:3 ratio of IPA:MTBE. After stirring at 16-17° C. for 3 days, the slurry was filtered, and the solids were washed with 12 L 1:3 IPA:MTBE. The solids were dried in vacuo (150 torr) at 50° C., with a nitrogen sweep through the batch, for 3 days. Zwitterion 1-9 was isolated as a white crystalline solid. Zwitterion 1-9 assays: 99.97 LCAP; >99.99% e.e.

¹H-NMR (400.25 MHz, D₂O) δ 7.30 (m, 1H), 6.92-6.85 (om, 2H), 4.68 (OH), 3.75-3.66 (om, 3H), 3.45 (bm, 1H), 3.30-3.14 (om, 2H), 1.32 (s, 9H).

¹³C-NMR (100.65 MHz, D₂O) δ 176.5, 162.8 (dd, J=123.7, 12.6), 160.3 (dd, J=124.5, 12.7), 129.9 (dd, J=10.1, 5.9), 119.7, 111.7 (dd, J=21.5, 3.6), 104.1 (t, J=26.2), 62.0, 51.9, 51.0, 50.6, 41.3, 23.7 (3C).

MP (DSC): onset 215° C., peak 217° C.

Anal. Calcd for C₁₅H₁₉F₂NO₂: Calc., C, 63.59; H, 6.76; F, 13.41; N, 4.94. Found, C, 63.50; H, 6.81; F, 13.11; N, 4.91.

### Preparation of (3S,4R)-1-tert-butyl-4-2,4-difluorophenyl)-pyrrolidine-3-carboxamide (1-8)

An analytical sample of the trans amide intermediate 1-8 was prepared from acid 1-9, via the acid chloride and quenching with ammonium hydroxide: ¹H NMR (CDCl₃) δ 7.28 (m, 1H), 6.84-6.73 (om, 2H), 6.60 (br s, H), 5.92 (br s, 1H), 3.67 (m, 1H), 3.26 (t, J=8.7, 1H), 3.08 (dd, J=9.2, 4.2, 11), 2.98 (t, J=8.3, 1H), 2.87 (m, 1H), 2.61 (t, J 8.5, 1H), 1.11 (s, 9H); ¹³C NMR (CDCl₃)δ 177.8, 161.7 (dd, J=248.4, 12.9), 160.7 (dd, J=248.6, 12.0), 129.8 (dd, J=9.4, 6.4), 126.1 (dd, J=14.1, 3.6), 111.4 (dd, J=20.9, 3.6), 104.0 (q, J=51.8), 53.2, 52.4, 51.2, 50.4, 41.5, 26.1 (3C). Anal. Calcd for C₁₅H₂₀F₂N₂₀: C, 63.81, H, 7.14, N, 9.92, F, 13.46, O 5.67. Found, C, 63.72, H, 7.00, N, 9.89, F, 13.91.

### EXAMPLE 2

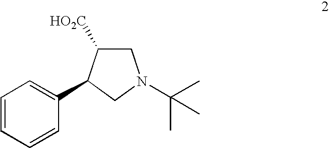

Compound 2 was prepared from 2-chloroacetophenone (Aldrich) following a similar procedure to that described for compound 1-9.

¹H-NMR (400.25 MHz, CD₃OD), δ 7.40 (m, 2H), 7.34 (m, 2H), 7.26 (m, 1H), 3.85 (m, 1H), 3.80-3.70 (m, 2H), 3.58 (br t, J=10.5, 1H), 3.31 (m, 1H), 3.16 (dd, J=18.8, 9.6, 1H), 1.43 (s, 9H). ¹³C-NMR (100.65 MHz, CD₃OD) δ 175.5, 138.0, 128.4, 127.3, 127.2, 61.1, 53.7, 52.3, 51.9, 47.4, 23.5. HR-MS M+H theoretical 248.1651; found 248.1649.

### EXAMPLE 3

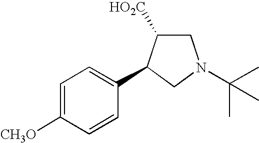

Compound 3 was prepared from 4′-methoxy-2-bromoacetophenone (Aldrich) following a similar procedure to that described for compound 1-9.

¹H-NMR (400.25 MHz, CD₃OD) δ 7.31 (d, J=8.7, 2H), 6.88 (d, J=8.7, 2H), 4.89 (OH), 3.79-3.68 (om, 3H), 3.76 (s, 3H), 3.55 (br t, J=10.6, 1H), 3.25 (br t, J=11.2, 1H), 3.11 (dd, J=18.8, 10.0, 1H), 1.41 (s, 9H).

¹³C-NMR (100.65 MHz, CD₃OD) δ 177.2, 160.7, 131.3, 129.9, 115.4, 62.6, 55.9, 55.2, 54.1, 53.3, 48.5, 25.0. HR-MS M+H theoretical 278.1756; found 278.1754.

1. A process for the preparation of compounds of structural formula (I): 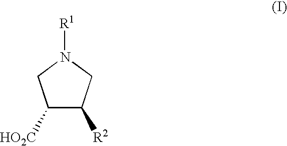
wherein
R¹ is selected from the group consisting of
(1) hydrogen,
(2) amidino,
(3) C₁₋₄ alkyliminoyl,
(4) C₁₋₁ alkyl,
(5) —(CH₂)ₙ—C₃₋₇ cycloalkyl,
(6) —(CH₂)ₙ-phenyl,
(7) —(CH₂)ₙ-naphthyl, and
(8) —(CH₂)ₙ-heteroaryl,
in which phenyl, naphthyl, and heteroaryl are unsubstituted or substituted with one to three groups independently selected from R³; and alkyl, cycloalkyl, and (CH₂)ₙ are unsubstituted or substituted with one to three groups independently selected from R³ and oxo;
R² is selected from the group consisting of
(1) C₁₋₄ alkyl,
(2) —(CH₂)ₙ-cycloalkyl,
(3) —(CH₂)ₙ-heterocycloalkyl,
(4) —(CH₂)ₙ-phenyl,
(5) —(CH₂)ₙ-naphthyl, and
(6) —(CH₂)ₙ-heteroaryl wherein heteroaryl is selected from the group consisting of
(1) pyridinyl,
(2) furyl,
(3) thienyl,
(4) pyrrolyl,
(5) oxazolyl,
(6) thiazolyl,
(7) imidazolyl,
(8) pyrazolyl,
(9) isoxazolyl,
(10) isothiazolyl,
(11) pyrimidinyl,
(12) pyrazinyl,
(13) pyridazinyl,
(14) quinolyl,
(15) isoquinolyl,
(16) benzimidazolyl,
(17) benzofuryl,
(18) benzothienyl,
(19) indolyl,
(20) benzthiazolyl, and
(21) benzoxazolyl;
in which alkyl, phenyl, naphthyl, heteroaryl, and (CH₂)ₙ are unsubstituted or substituted with one to three groups independently selected from R³;
each R³ is independently selected from the group consisting of
(1) C₁₋₆ alkyl,
(2) —(CH₂)ₙ-phenyl,
(3) —(CH₂)ₙ-naphthyl,
(4) —(CH₂)ₙ-heteroaryl,
(5) —(CH₂)ₙ-heterocycloalkyl,
(6) —(CH₂)ₙC₃₋₇ cycloalkyl,
(7) halogen,
(8) OR⁴,
(9) —(CH₂)ₙN(R⁴)₂,
(10) NO₂,
(11) —(CH₂)ₙNR⁴SO₂R⁴,
(12) —(CH₂)ₙSO₂N(R⁴)₂,
(13) —(CH₂)ₙS(O)ₚR⁴,
(14) CF₃,
(15) CH₂CF₃,
(16) OCF₃, and
(17) OCH₂CF₃;
in which heteroaryl is as defined above; alkyl, phenyl, naphthyl, heteroaryl, cycloalkyl, and heterocycloalkyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of
(1) hydrogen,
(2) C₁₋₆ alkyl,
(3) —(CH₂)ₙ-phenyl,
(4) —(CH₂)ₙ-heteroaryl,
(5) —(CH₂)ₙ-naphthyl,
(6) —(CH₂)ₙ-heterocycloalkyl,
(7) —(CH₂)ₙC₃₋₇ cycloalkyl, and
(8) —(CH₂)ₙC₃₋₇ bicycloalkyl;
wherein alkyl, phenyl, heteroaryl, heterocycloalkyl, and cycloalkyl are unsubstituted or substituted with one to three groups independently selected from halogen, C₁₋₄ alkyl, hydroxy, and C₁₋₄ alkoxy;
or two R⁴ groups together with the atom to which they are attached form a 4- to 8-membered mono- or bicyclic ring system optionally containing an additional heteroatom selected from O, S, and NC₁₋₄ alkyl; and
n is 0, 1, 2, 3 or 4;
comprising the steps of:
(a) preparing an alcohol of structural formula (V) 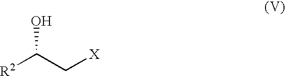
wherein
X is bromide or chloride, and R² is as defined above,
by treating a ketone of structural formula (IV), 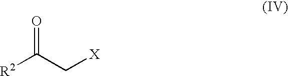
wherein X is bromide or chloride, and R² is as defined above, with a reducing agent, and isolating the resulting product;
(b) forming an amino alcohol of structural formula (VII) 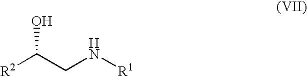
wherein R¹ and R² are as defined above,
by treating the alcohol of structural formula (V) with an amine of general formula R¹NH₂, wherein R¹ is as defined above, and a base in a solvent, and isolating the resulting product;
(c) forming a compound of structural formula (VIII) 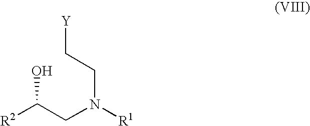
wherein Y is —CN or —CO₂R⁵ and R⁵ is C₁₋₄ alkyl and wherein R¹ and R² are as defined above,
by treating the amino alcohol of structural formula (VU) with a compound of general formula (XI) 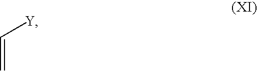
wherein Y is —CN or —CO₂R⁵, and R⁵ is C₁₋₄ alkyl, and isolating the resulting product;
(d) forming a pyrrolidine compound of structural formula (X) 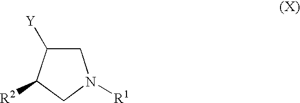
wherein Y, R¹ and R² are as defined above,
by treating the compound of structural formula (VI) with an alcohol activating reagent, followed by a base;
(e) forming a trans-pyrrolidine acid of structural formula (I) 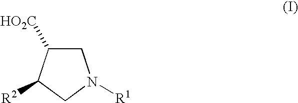
wherein R¹ and R² are as defined above,
by hydrolyzing the pyrrolidine compound of structural formula (X) with an aqueous base in a solvent; and
(f) isolating the resulting product.

2. The process of claim 1 wherein the reducing agent used to treat compound of formula (IV) of step (a) is (+)-DIP chloride.

3. The process of claim 1 wherein the compound of formula (IV) of step (a) is treated with a reducing agent selected from the group consisting of borane-N,N-diethyl aniline, borane-THF, and borane-dimethylsulfide, in the presence of a catalyst.

4. The process of claim 3 wherein the reducing agent is borane-N,N-diethyl aniline.

5. The process of claim 4 wherein the catalyst selected from the group consisting of (S)-CBS and (S)-2-methyl CBS oxazaborolidine.

6. The process of claim 5 wherein the catalyst is (S)-2-methyl CBS oxazaborolidine.

7. The process of claim 1 wherein the alcohol of formula (V) is treated with an amine of general formula R¹NH₂, wherein R¹ is selected from the group consisting of hydrogen, —(CH₂)ₙphenyl, and C₁₋₆alkyl.

8. The process of claim 7 wherein R¹ is tert-butyl.

9. The process of claim 1 wherein the alcohol of formula (V) is treated with a base selected from the group consisting of NaOH, LiOH, and KOH.

10. The process of claim 9 wherein the base is NaOH.

11. The process of claim 1 wherein, the compound of formula (XI) is the compound wherein Y is —CN.

12. The process of claim 11 wherein the compound of formula (VIII) is formed by adding a 1:1 mixture of ethanol: formamide.

13. The process of claim 1 wherein the amino alcohol of formula (VIII) is treated with an alcohol activating reagent selected from the group consisting of ClPO(OR⁶)₂, ClPO(N(R⁶)₂)₂, MsCl, Ms₂O, TsCl, and Ts₂O, wherein R⁶ is C₁₋₄ alkyl or phenyl.

14. The process of claim 13 wherein the alcohol activating reagent is chlorodiethyl phosphate.

15. The process of claim 1 wherein amino alcohol of formula (VI) is treated with a base selected from the group consisting of lithium hexamethyl disilazide, sodium hexamethyl disilazide, and potassium hexamethyldisilazide.

16. The process of claim 15 wherein the base is lithium hexamethyl disilazide.

17. The process of claim 1 wherein the pyrrolidine compound of formula (X) is hydrolyzed with a base selected from the group consisting of NaOH, LiOH and KOH.

18. The process of claim 17 wherein the base is NaOH.

19. The process of claim 1 wherein R² is phenyl or thienyl optionally substituted with one to three groups independently selected from R³.

20. The process of claim 19 wherein R² is phenyl optionally substituted with one to three groups independently selected from R³.

21. The process of claim 20 wherein R³ is selected from the group consisting of halogen, —CF₃, and OR⁴, wherein R⁴ is as defined in claim 1.

22. The process of claim 21 wherein R² is selected from the group of phenyl; ortho, para-difluorophenyl; and para-methoxyphenyl.

23. The process of claim 22 wherein R² is ortho, para-difluorophenyl.

24. The process of claim 1 wherein the compound of structural formula (I) is isolated by forming a zwitterion of the trans pyrrolidine acid of structural formula (I) 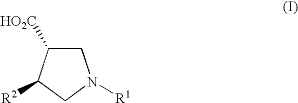
wherein R¹ and R² are as defined above; recrystallizing the zwitterion from a solvent; and isolating the resulting product.

25. The process of claim 24 wherein the zwitterion of the pyrrolidine acid of formula (I) is formed at the isoelectric pH using an acid.

26. The process of claim 25 wherein the acid is selected from sulfuric acid or hydrochloric acid.

27. The process of claim 26 wherein the acid is sulfuric acid.

28. The process of claim 24 wherein the zwitterion of the pyrrolidine acid of formula (I) is recrystallized from a solvent.

29. The process of claim 28 wherein the solvent is selected from the group consisting of ethanol, isopropyl alcohol, methyl tert-butyl ether or a mixture thereof.

30. The process of claim 29 wherein the solvent is a mixture of 1:3 isopropyl alcohol:methyl tert-butyl ether.

31. (canceled)

32. The compound 2 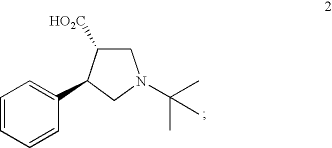
or a zwitterion or salt thereof.

33. The compound 3 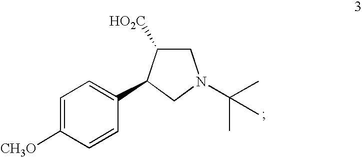
or a zwitterion or a salt thereof.

34. A process for the preparation of compounds of structural formula (I): 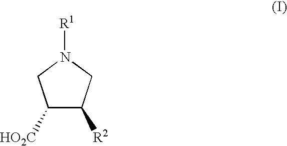
wherein
R¹ is selected from the group consisting of
(1) hydrogen,
(2) amidino,
(3) C₁₋₄ alkyliminoyl,
(4) C₁₋₁₀ alkyl,
(5) —(CH₂)ₙ—C₃₋₇ cycloalkyl,
(6) —(CH₂)ₙ-phenyl,
(7) —(CH₂)ₙ-naphthyl, and
(8) —(CH₂)ₙ-heteroaryl,
in which phenyl, naphthyl, and heteroaryl are unsubstituted or substituted with one to three groups independently selected from R³; and alkyl, cycloalkyl, and (CH₂)ₙ are unsubstituted or substituted with one to three groups independently selected from R³ and oxo;
R² is selected from the group consisting of
(1) C₁₋₄ alkyl,
(2) —(CH₂)ₙ-cycloalkyl,
(3) —(CH₂)ₙ-heterocycloalkyl,
(4) —(CH₂)ₙ-phenyl,
(5) —(CH₂)ₙ-naphthyl, and
(6) —(CH₂)ₙ-heteroaryl wherein heteroaryl is selected from the group consisting of
(1) pyridinyl,
(2) furyl,
(3) thienyl,
(4) pyrrolyl,
(5) oxazolyl,
(6) thiazolyl,
(7) imidazolyl,
(8) pyrazolyl,
(9) isoxazolyl,
(10) isothiazolyl,
(11) pyrimidinyl,
(12) pyrazinyl,
(13) pyridazinyl,
(14) quinolyl,
(15) isoquinolyl,
(16) benzimidazolyl,
(17) benzofuryl,
(18) benzothienyl,
(19) indolyl,
(20) benzthiazolyl, and
(21) benzoxazolyl;
in which alkyl, phenyl, naphthyl, heteroaryl, and (CH₂)ₙ are unsubstituted or substituted with one to three groups independently selected from R³;
each R³ is independently selected from the group consisting of
(1) C₁₋₆ alkyl,
(2) —(CH₂)ₙ-phenyl,
(3) —(CH₂)ₙ-naphthyl,
(4) —(CH₂)ₙ-heteroaryl,
(5) —(CH₂)ₙ-heterocycloalkyl,
(6) —(CH₂)ₙC₃₋₇ cycloalkyl,
(7) halogen,
(8) OR⁴,
(9) —(CH₂)ₙN(R⁴)₂,
(10) NO₂,
(11) —(CH₂)ₙNR⁴SO₂R⁴,
(12) —(CH₂)ₙSO₂N(R⁴)₂,
(13) —(CH₂)ₙS(O)ₚR⁴,
(14) CF₃,
(15) CH₂CF₃,
(16) OCF₃, and
(17) OCH₂CF₃;
in which heteroaryl is as defined above; alkyl, phenyl, naphthyl, heteroaryl, cycloalkyl, and heterocycloalkyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of
(1) hydrogen,
(2) C₁₋₆ alkyl,
(3) —(CH₂)ₙ-phenyl,
(4) —(CH₂)ₙ-heteroaryl,
(5) —(CH₂)ₙ-naphthyl,
(6) —(CH₂)ₙ-heterocycloalkyl,
(7) —(CH₂)ₙC₃₋₇ cycloalkyl, and
(8) —(CH₂)ₙC₃₋₇ bicycloalkyl;
wherein alkyl, phenyl, heteroaryl, heterocycloalkyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl; and
n is 0, 1, 2, 3 or 4;
comprising the steps of:
(a) hydrolyzing a pyrrolidine compound of structural formula (X), wherein Y is —CN or —CO₂R⁵ and R⁵ is C₁₋₄ alkyl, and wherein R¹ and R² are as defined above, 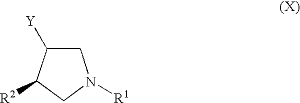
with an aqueous base in a solvent; and
(b) isolating the resulting product.

35. The process of claim 34 wherein the pyrrolidine compound of formula (X) is hydrolyzed with a base selected from the group consisting of NaOH, LiOH and KOH.

36. The process of claim 35 wherein the base is aqueous NaOH.

37. The process of claim 36 wherein R² is selected from the group of phenyl; ortho, para-difluorophenyl; and para-methoxyphenyl.

38. The process of claim 37 wherein R² is ortho, para-difluorophenyl.

39. The process of claim 34 wherein R¹ is tert-butyl.
